(19) United States
(12) Patent Application Publication
(10) Pub. No.: US 2010/0191157 A1
(43) Pub. Date: Jul. 29, 2010

(54) Method for treating skin lesions

(76) Inventor: Narendra T. Sanghvi, Indianapolis, IN (US)

Correspondence Address: REED SMITH LLP, 2500 ONE LIBERTY PLACE, 1650 MARKET STREET, PHILADELPHIA, PA, 19103, US

(21) Appl. No.: 12/322,066

(22) Filed: Jan. 27, 2009

(51) Int. Cl. A61N 7/00; A61M 37/00

(52) U.S. Cl. 601/2; 604/22

(57) ABSTRACT

A method for treatment of skin lesions is provided using High Intensity Focused Ultrasound (HIFU). The method involves administering HIFU to a targeted skin lesion, wherein the application of the HIFU results in the removal of the targeted skin lesion and also results in the removal of skin lesions that are not directly targeted by the application of HIFU.

The present invention relates to a method for the treatment and removal of skin lesions.

### BACKGROUND

Skin lesions such as warts and actinic keratoses commonly affect people of all ages. They can be painful, an object of ridicule, and can lead to a loss of confidence over appearance. Additionally, skin lesions can pose public health and safety concerns, and as a result, many patients demand timely and effective treatment. Some skin lesions, such as actinic keratosis, can be pre-cancerous and can transform into malignancies such as squamous cell carcinoma.

One approach to skin lesion treatment involves the application of salicylic acid to the lesion, typically a wart. The salicylic acid erodes the wart. Salicylic acid, however, can severely irritate the patient's skin, which can cause painful side effects. Liquid nitrogen cyrotherapy treatment can be used to treat skin lesions such as warts or actinic keratoses. The application of liquid nitrogen freezes the lesion so that it becomes necrotic and falls off the patient's skin. Liquid nitrogen cyrotherapy, however, can cause pain and discomfort in the treated region.

Both salicylic acid treatment and liquid nitrogen cyrotherapy treatment require application to one lesion at a time. For example, if salicylic acid and liquid nitrogen are used to treat warts, the treatment must be conducted on a wart-by-wart basis. These treatments can also change the appearance of the skin to which it is applied, thus furthering the patient's loss of confidence over appearance.

Another approach to treatment of skin lesions, particularly actinic keratosis treatment, involves the application of a topical 5-fluorouracil ointment to the skin of patients with extensive actinic keratosis. This treatment, however, requires repeated application of the ointment for up to four weeks. Additionally, this treatment can cause pain and inflammation on the patient's skin. Preferably, a treatment which requires a less repetitive application and which does not result in skin inflammation is desired.

Yet another approach to treatment of skin lesions is the use of photodynamic therapy. A photodynamic agent, such as aminolevulinic acid, is applied to the skin and becomes localized in the lesion, whereupon it is activated by a suitable wavelength of light. See, e.g. U.S. Pat. No. 5,422,093, herein incorporated by reference.

Yet another approach to treatment of skin lesions, particularly wart treatment, involves injection of a therapeutic agent, such as bleomycin, into the wart itself. This treatment can cause great pain in the region of the bleomycin injection. Preferably, a minimally invasive and less painful treatment mechanism than bleomycin injection is desired.

Yet another approach to wart treatment involves the combination of electrosensitization and ultrasound to treat the targeted tissue. An important disadvantage associated with this method is that it requires treatment on a wart-by-wart basis. This treatment is not effective in treating unidentified warts or warts which are distant from the region of skin treated by the electrosensitization and ultrasound combination. A wart treatment, which does not require the selective treatment of each individual wart on a wart-by-wart basis would be preferred over this electrosensitization and ultrasound combination technique. Another major disadvantage associated with the use of this combination method is that it requires the use of two separate pieces of equipment, one for electrosensitization and one for ultrasound application. The need for two separate pieces of equipment greatly adds to the cost and time required for administering this combination treatment. A wart treatment, which does not require the use of multiple pieces of equipment, which is less costly, and which requires less time to be administered, is preferred over the electrosensitization and ultrasound combination technique. Thus, the present invention provides an advantage over the electrosensitization and ultrasound combination technique.

Thus, a skin lesion treatment which causes little or no pain, requires few or no repeated applications, does not disfigure the treatment area, and does not require the selective treatment of each individual lesion is preferred over salicylic acid and liquid nitrogen cyrotherapy treatment.

### SUMMARY OF THE INVENTION

It has been found that the application of Low Energy, High Intensity Focused Ultrasound (LO-HIFU) and High Energy, High Intensity Focused Ultrasound (HI-HIFU) can selectively treat a target lesion while simultaneously eliciting beneficial immunotherapy responses to treat remote lesions.

The present invention provides a method for treating skin lesions on a patient through direct application of HIFU to the lesion. The lesion (referred to as “targeted lesion”) is subjected to LO-HIFU and HI-HIFU, and as a result of such treatment, the targeted lesion is removed. Alternatively, the lesion is subjected to only HI-HIFU, and after such treatment, the targeted lesion is removed.

The present invention also provides a method for treating skin lesions that are not the targeted lesion by eliciting an immune response. When a targeted lesion is subjected to LO-HIFU and HI-HIFU this HIFU treatment of the targeted lesion triggers an immune response such that the lesions that are not the targeted lesion are no longer present on the patient's skin. The lesion is destroyed at the time of treatment and healing takes a few days.

Alternatively, a targeted lesion is subjected to only HI-HIFU. The HI-HIFU treatment of the targeted lesion triggers an immune response such that those lesions that are not the targeted lesion are no longer present on the patient's skin.

The present invention further provides a coupling apparatus that couples the transducer generating the HIFU to the targeted lesion. The apparatus acts to couple the HIFU transducer to the skin lesion such that the apparatus focuses the ultrasound emitted from the HIFU transducer onto the skin lesion. The apparatus preferably has a geometric shape to allow for a precise location of the transducer related to the lesion to be targeted by the HIFU transducer.

As used herein, the term “remove” or “removal” means the eventual substantial disappearance of a skin lesion from a patient's skin.

Additional features of the present invention will become apparent to those skilled in the art upon review of the drawings and the detailed descriptions.

### DETAILED DESCRIPTION OF THE INVENTION

Non-invasive ultrasound treatments have been well established as effective for tissue removal. Specifically, HIFU techniques are known to remove and treat selectively unwanted tissue in the body. HIFU technology allows for a high intensity ultrasound emitter to focus an acoustic beam onto a targeted tissue on the body. Depending on the applied energy level, the duration of application, and the repetition frequency, the target tissue can undergo mechanical and/or thermal disruption.

The HIFU applied to a targeted skin lesion can be classified in two types: Low Energy HIFU (LO-HIFU) and High Energy HIFU (HI-HIFU).

HI-HIFU treatment is defined as a treatment results in the necrosis or ablation of the targeted lesion. HI-HIFU typically applies 3.1 to 9.1 KW-sec of energy per cm², though intensities higher and lower than this range can be used. HI-HIFU typically operates with a continuous wave applied for a duration in the range of 3 to 20 seconds and with an operating frequency range between 1 and 5 MHz, though longer and shorter durations can be used with higher or lower operation frequencies. When HI-HIFU is used to cause extensive or essentially complete necrosis, the frequency of the HIFU can be greater than about 20 KHz and less than about 100 MHz. An example of a HI-HIFU treatment is the complete ablation of the prostate using HI-HIFU with the result of the entire prostate experiencing coagulative necrosis.

In contrast, LO-HIFU treatment is a treatment with energy levels such that the targeted lesion is agitated but remains viable. Pulsing is a way of modulating power, and LO-HIFU can conveniently be accomplished by pulsing of a HI-HIFU source. LO-HIFU then can be accomplished by moving a HIFU transducer to move the focus of the HIFU before sufficient energy is applied to result in necrosis or ablation. In one embodiment HI-HIFU is swept across the targeted tissue to accomplish LO-HIFU. LO-HIFU typically applies 0.01 to 1.0 KW-sec of energy per cm², though intensities higher and lower than this range can be used. LO-HIFU, when applied in a pulsed manner, conveniently operates with a pulse duration in the 1 to 100 millisecond range with pulse repetition frequencies in the range between 0.5 to 5.0 Hz, though longer and shorter pulse durations can be used with higher or lower repetition frequencies. In one embodiment, LO-HIFU operates with a repetition frequency in the range between 0.5 to 30 Hz. An example of a LO-HIFU treatment is the application of LO-HIFU in order to disturb the blood-brain barrier to permit the passage of drugs from the blood stream into the brain.

The present invention involves a method using a combination of LO-HIFU and HI-HIFU to treat skin lesions. Such skin lesions can be located anywhere on a patient's body. Such skin lesions can be located on the fingers, toes, face, eyes, nose and mouth of a patient. The LO-HIFU/HI-HIFU combination is effective in direct treatment of a lesion in the skin region where the HIFU combination is focused. The LO-HIFU/HI-HIFU combination is also effective in treatment of lesions which are different than the skin region where the HIFU combination is focused.

For the direct treatment of a skin lesion in the region where the HIFU combination is directly focused, the application of the LO-HIFU and HI-HIFU combination induces coagulative necrosis of the targeted lesion. The acoustic beams from the HIFU combination are delivered to the targeted skin lesion by a high intensity ultrasound emitter. The acoustic beams emanating from the high intensity ultrasound emitter are focused onto the targeted skin region through the use of a coupler apparatus. The coupler apparatus has a geometrical shape configured to make contact with a precise tissue location so that the acoustic beams from the ultrasound emitter are delivered only to that precise tissue location. Preferably, the geometric shape of the coupling apparatus is conical, such that the bottom portion has a large surface area to make contact with the ultrasound emitter and that the top portion has a narrow surface area to make contact only with a precise tissue location.

The present invention involves a method to treat skin lesions different than those lesions that are not the targeted lesion by eliciting an immune response. When a targeted lesion is subjected to LO-HIFU and/or HI-HIFU, this HIFU treatment of the targeted lesion triggers an immune response such that the lesions that are not the targeted lesion are no longer present on the patient's skin. A LO-HIFU treatment includes the administration of acoustic energy in a manner that causes a membrane of a cell to be disrupted with some associated hyperthermia while maintaining cell viability and avoiding cavitations. The application of LO-HIFU allows for cellular molecules to pass through the cell membrane as discussed in the article by K M Dittmar, J Xie, F Hunter, C Trible, M Bur, V Frenkel, and K C P Li, titled “Pulsed High-Intensity Focused Ultrasound Enhances Systemic Administration of Naked DNA in Squamous Cell Carcinoma Model: Initial Experience,” herein incorporated by reference. The application of HIFU induces heat shock proteins within the skin lesion. These heat shock proteins are released in the blood after HIFU treatment of the lesions. Since HSPs-bind to peptides, spontaneous release of HSPs from HIFU-treated lesion cells should provide a source of antigens for antigen presentation by circulating dendritic cells (DCs). The cellular material 308 is expected to be uptaken by the DCs. The antigen-loaded DCs are then expected to migrate into the lymph nodes. Furthermore, the HSPs released in the blood after HIFU treatment of a skin lesion are expected to provide the “danger” signals to the DCs and also to provide a source of comprehensive peptides for efficient antigen presentation.

In the lymph nodes, maturation of the DCs occurs. In one example, CD40L provides signals for DC maturation and thereby eliminates the need of CD4 T cells. Further, in one example, secondary lymphoid chemokines (SLCs) assist in the migration of DCs into draining lymph nodes and interact with T cells to induce a cell-mediated anti-lesion immunity. Sequential use of T lymphocyte-stimulating cytokines induces a strong immune response. Further, IL-2 amplifies the lesion-specific cytotoxic T lymphocytes and IL-15 induces a strong memory T cell response.

As a result, cytotoxic T lymphocytes (CTLs) proliferate and destroy other skin lesion cells. Thus, HIFU-treated lesion cells serve as an in situ lesion vaccine and induce a strong immune response to eradicate distant skin lesions.

In one embodiment, administration of anti-CTLA4 antibodies after the HIFU treatment down-regulate regulatory T cells, thereby augmenting the lesion-specific immune response. In another embodiment, administration of 4-1BBL enhances the CTL effector and memory T cells. In still another embodiment, a variety of CpG oligonucleotides are used with the HIFU treatment to enhance the innate immunity after treatment. CpG oligonucleotides induce DC maturation and break tolerance to lesion antigens.

Instead of individualized vaccines, the immunotherapy treatment depends on the endogenous circulating DCs to harvest the lesion antigens released by dying cells after an HIFU treatment. In addition, combining traditional immunotherapy with local skin lesion HI-HIFU and/or LO-HIFU treatment should at least allow the local energy treatment to reduce the lesion burden without causing generalized immuno-suppression as seen with chemotherapy. Further, the combination should also serve as antigen depot for “boosting” immunological memory and inducing a more extensive immune response by antigen spreading, thereby enhancing the vaccine effect.

This embodiment produced unexpected results. When skin lesions were treated by direct HIFU treatment, other skin lesions, which were located distant from the region of treatment, were removed. This result was possibly due to the immune response by this embodiment, but these results are not limited to this immune response.

### EXAMPLES

The following examples demonstrate the embodiment of the present invention as described above.

The present invention will be further illustrated with reference to the following examples. It will be appreciated that what follows is by way of example only and that modifications to detail may be made while still falling within the scope of the invention.

### Example 1

In the first example, HI-HIFU was directly applied to warts on two human patients. The HIFU was administered with an energy intensity between 3.1 to 9.1 KW-sec of energy per cm² and with an operating frequency between 1.0 to 5.0 MHz. The HIFU was being administered for a duration between 3 to 20 seconds.

The warts that were subjected to direct HI-HIFU treatment were removed within days. Subsequently, warts that were not subjected to direct HI-HIFU were removed later, typically within nine to twelve months.

### Example 2

In the second example, a combination of HI-HIFU and LO-HIFU is being applied directly to warts on a patient. LO-HIFU is being applied before HI-HIFU. The HI-HIFU is being applied with an energy intensity between 3.1 to 9.1 KW-sec of energy per cm² and with an operating frequency between 1.0 to 5.0 MHz. The LO-HIFU is being accomplished by pulsing the HI-HIFU with a pulse repetition frequency range between 0.5 to 5.0 Hz and with a pulse duration between 1 to 100 milliseconds. The LO-HIFU is being administered for a duration between 0.01 to 1.0 seconds, with an application of 20 pulse repetitions.

The warts that are subjected to the direct treatment with a HI-HIFU and LO-HIFU combination will be removed shortly after the HIFU is administered. Generally, the warts that are subjected to the direct treatment with a HI-HIFU and LO-HIFU combination will be removed within ten to thirty days after the HIFU is administered. Subsequently, warts that are not subjected to direct HIFU treatment will be removed later, typically within nine to twelve months after the HIFU is administered.

### Example 3

The third example involves administering drugs to boost a patient's immune response after HIFU is administered. The immune response provoked by the use of HIFU is enhanced by the administration of compositions which cause the body to have a stronger immune response. These immune boosting compositions are known in the art, although their use in the context of HIFU treatments is novel. Examples of these compositions include cytokines, such as lymphokines and chemokines. Preferably, these are dendritic cell stimulating cytokines. These immune boosting compositions include G-CSF, GM-CSF, IL-4, and Fit3L. A number of suitable immune boosting compositions are available commercially and are already approved for use in humans, though not yet approved for use in this context. For example, Leukine is a GM-CSF product available from Berlex Laboratories. Neupogen is recombinant methionyl human granulocyte colony-stimulating factor (r-metHuG-CSF) available from Amgen. Neulasta is a covalent conjugate of recombinant methionyl human G-CSF and monomethoxypolyethylene glycol and is also available from Amgen. Proleukin is IL-2, and is available from Novartis. Other useful immune boosting compositions include IL-2, IL-I-5, CD40L, 4-1 BB ligand, or a CpG oligonucleotide. These and other immune boosting compositions are effective in enhancing the immune response provoked by use of HIFU according to the present invention. Techniques for their safe use are well known to those skilled in the cancer therapy field. The specific dose of each of these compositions which is used in a particular patient is a matter for the well-informed clinician's professional judgment based on well established factors including body weight, prior status of the patient's immune system, and the closely monitored response of the patient to the composition. This same judgment, operating within these well established clinical parameters, applies when the immune boosting compositions are used as part of this invention.

In this example, HIFU is applied directly to warts on a patient. After such application, the patient is treated with immune boosting drugs. The warts that are subjected to direct treatment of HIFU will be removed shortly after the HIFU is administered, generally between ten to thirty days. Subsequently, warts that are not subjected to direct HIFU treatment are removed later, typically within nine to twelve months after the direct HIFU treatment.

1. A method for treatment of a skin lesion, the method comprising the steps of
administering HIFU to a targeted skin lesion, wherein the application of the HIFU results in the removal of the targeted skin lesion.

2. The method of claim 1, wherein the HIFU is a high energy, high intensity focused ultrasound (HI-HIFU).

3. The method of claim 1, wherein the HIFU is a low energy, high intensity focused ultrasound (LO-HIFU).

4. The method of claim 1, wherein the HIFU is administered as a combination of HI-HIFU and LO-HIFU.

5. The method of claim 2, wherein the targeted skin lesion is subjected to HI-HIFU that is applied continuously for about 3 to 20 seconds.

6. The method of claim 3, wherein the targeted skin lesion is subjected to LO-HIFU that is applied as a pulse with a pulse duration of about 1 to 100 milliseconds.

7. The method of claim 3, wherein the targeted skin lesion is subjected to LO-HIFU by moving a HIFU focus across the skin lesion.

8. The method of claim 2, wherein the targeted skin lesion is subjected to HI-HIFU that moves across the skin lesion.

9. The method of claim 1, wherein the HIFU causes necrosis of the lesion.

10. The method of claim 1, wherein the HIFU does not cause necrosis of the lesion.

11. The method of claim 1, wherein the HIFU causes necrosis of a portion of the lesion.

12. The method of claim 2, wherein the HI-HIFU applies energy in the amount of about 3.1 to 9.1 KW-sec per cm².

13. The method in claim 2, wherein the administration of HI-HIFU operates continuously for about 3 to 20 seconds and with an operating frequency of about 1.0 to 5.0 MHz.

14. The method of claim 2, wherein the HI-HIFU has an operating frequency greater than about 20 KHz and less than about 100 MHz.

15. The method of claim 3, wherein the LO-HIFU applies energy in the amount of about 0.01 to 3.0 KW-sec per cm².

16. The method of claim 3, wherein the LO-HIFU operates with a pulse having a duration of about 0.01 to 1.0 seconds with pulse repetition frequencies of about 0.5 to 5.0 Hz.

17. The method of claim 3, wherein the LO-HIFU operates with repetition frequencies of about 0.5 to 30.0 Hz.

18. The method of claim 8, wherein the HI-HIFU operates with repetition frequencies of about 0.5 to 30.0 Hz.

19. The method of claim 1, wherein the HIFU is applied in one of the following approaches: extracorporeal, infra cavity, percutaneous, robotic, laparoscopic, and directly on the tumor.

20. The method of claim 1, wherein the HIFU is applied using one or more of intensity modulation and frequency modulation.

21. The method of claim 6, wherein the frequency of the HIFU is greater than about 20 KHz and less than about 100 MHz.

22. A method for treatment of a skin lesion, the method comprising the steps of
administering HI-HIFU to the targeted skin lesion;
wherein the application of HI-HIFU results in the removal of the targeted lesion.

23. The method of claim 1, wherein the targeted skin lesion is subjected to HI-HIFU that moves across the skin lesion.

24. The method of claim 1, wherein the HI-HIFU applies energy in the amount of about 3.1 to 9.1 KW-sec per cm².

25. The method of claim 1, wherein the HI-HIFU operates continuously for about 3 to 20 seconds with an operating frequency of about 1.0 to 5.0 MHz.

26. The method of claim 2, wherein the HI-HIFU has an operating frequency greater than about 20 KHz and less than about 100 MHz.

27. The method of claim 23, wherein the HI-HIFU operates with repetition frequencies of about 0.5 to 30.0 Hz.

28. A method for treatment of multiple skin lesions, the method comprising the steps of
administering LO-HIFU to targeted skin lesions;
administering HI-HIFU to the targeted skin lesions;
wherein, the administration of LO-HIFU and the administration of HI-HIFU results the targeted skin lesions being removed; and
wherein, the administration of LO-HIFU and the administration of HI-HIFU results in skin lesions that are not the targeted skin lesions being removed.

29. The method of claim 28, wherein the removal of skin lesions that are not the targeted skin lesions is caused by an immune response that is provoked by the administration of HIFU to the targeted lesion.

30. The method in claim 28, wherein the administration of LO-HIFU applies energy in the amount of about 0.01 to 3.0 kilowatts-seconds per cm² to the lesions.

31. The method of claim 28, wherein the targeted skin lesion is subjected to LO-HIFU by moving a HIFU focus across the skin lesion.

32. The method of claim 28, wherein the LO-HIFU applies energy in the amount of about 0.01 to 3.0 KW-sec per cm².

33. The method of claim 28, wherein the LO-HIFU operates with a pulse having a duration of about 0.01 to 1.0 seconds with pulse repetition frequencies of about 0.5 to 5.0 Hz.

34. The method of claim 28, wherein the LO-HIFU operates with repetition frequencies of about 0.5 to 30.0 Hz.

35. The method in claim 28, wherein the administration of HI-HIFU applies energy in the amount of about 3.1 to 9.1 kilowatts-seconds per cm² to the lesions.

36. The method in claim 28, wherein the administration of HI-HIFU operates continuously for about 3 to 20 seconds and with an operating frequency of about 1.0 to 5.0 MHz.

37. The method of claim 28, wherein the HI-HIFU has an operating frequency greater than about 20 KHz and less than about 100 MHz.

38. A method for treatment of multiple skin lesions, the method comprising the steps of
administering HI-HIFU to targeted skin lesions;
wherein, administration of HI-HIFU results in the removal of the targeted skin lesions; and
wherein, the administration of HI-HIFU results in the removal of skin lesions that are not the targeted skin lesions.

39. The method of claim 38, wherein the administration of HI-HIFU provokes an immune response that causes skin lesions that are not the targeted skin lesions to be removed.

40. The method in claim 38, wherein the administration of HI-HIFU applies energy in the amount of about 3.1 to 9.1 kilowatts-seconds per cm² to the lesions.

41. The method in claim 38, wherein the administration of HI-HIFU operates continuously for about 3 to 20 seconds and with an operating frequency of about 1.0 to 5.0 MHz.

42. The method of claim 38, wherein the HI-HIFU has an operating frequency greater than about 20 KHz and less than about 100 MHz.

43. A method for treatment of skin lesions, the method comprising the steps of
administering LO-HIFU to a targeted skin lesion in a patient;
administering HI-HIFU to the targeted skin lesion to cause the targeted skin lesion to release cellular material;
wherein the administration of HIFU stimulates the patient's immune response to the targeted skin lesion.

44. The method in claim 43 further comprising the step of administering an immune boosting drug in order to enhance the patent's immune response.

45. The method in claim 43, wherein the immune boosting drug is a G-CSF.

46. The method in claim 43, wherein the immune boosting drug is a GM-CSF.

47. The method in claim 43, wherein the immune boosting drug is a IL-4.

48. The method in claim 43, wherein the immune boosting drug is a Fit3L.

49. The method of claim 43, wherein the LO-HIFU applies energy in the amount of about 0.01 to 3.0 KW-sec per cm².

50. The method of claim 43, wherein the LO-HIFU operates with a pulse having a duration of about 0.01 to 1.0 seconds with pulse repetition frequencies of about 0.5 to 5.0 Hz.

51. The method of claim 43, wherein the LO-HIFU operates with repetition frequencies of about 0.5 to 30.0 Hz.

52. The method in claim 43, wherein the administration of HI-HIFU applies energy in the amount of about 3.1 to 9.1 kilowatts-seconds per cm² to the lesions.

53. The method in claim 43, wherein the administration of HI-HIFU operates continuously for about 3 to 20 seconds and with an operating frequency of about 1.0 to 5.0 MHz.

54. The method of claim 43, wherein the HI-HIFU has an operating frequency greater than about 20 KHz and less than about 100 MHz.

55. A method for treatment of skin lesions, the method comprising the steps of
administering HI-HIFU to a targeted skin lesion in a patient to cause the targeted skin lesion to release cellular material;
wherein the administration of HI-HIFU provokes the patient's immune response to the targeted skin lesion.

56. The method in claim 55 farther comprising the step of administering an immune boosting drug in order to enhance the patent's immune response.

57. The method in claim 55, wherein the immune boosting drug is a G-CSF.

58. The method in claim 55, wherein the immune boosting drug is a GM-CSF.

59. The method in claim 55, wherein the immune boosting drug is a IL-4.

60. The method in claim 55, wherein the immune boosting drug is a Fit3L.

61. The method in claim 55, wherein the administration of HI-HIFU applies energy in the amount of about 3.1 to 9.1 kilowatts-seconds per cm² to the lesions.

62. The method in claim 55, wherein the administration of HI-HIFU operates continuously for about 3 to 20 seconds and with an operating frequency of about 1.0 to 5.0 MHz.

63. The method of claim 55, wherein the HI-HIFU has an operating frequency greater than about 20 KHz and less than about 100 MHz.

64. An apparatus for focusing an ultrasound beams, which comprises
a coupling device with a top end and a bottom end, the top end placed adjacent to an ultrasound emitting device, the bottom end placed adjacent to location where the ultrasound beams are to be focused;
a coupling material, the material acting as an acoustic coupler to allow the ultrasound beams to travel from the ultrasound emitting device to a location where the beams are to be focused;
wherein the bottom end has a geometric configuration that directs the ultrasound energy to a skin lesion.

65. The apparatus in claim 64, wherein the apparatus has a conical shape.
